SEQUENCE LISTING (4 sequences)

SEQ ID NO: 1 - DNA, 51 bp, Homo sapiens
ctcctcgccccaggactctacacccrggacggcaatgctgatgggaaaccc

SEQ ID NO: 2 - DNA, 51 bp, Homo sapiens
aggtgaaagtcaggcatcagtattcwagagaaaatcctgagtgattccaat

SEQ ID NO: 3 - DNA, 52 bp, Homo sapiens
agatgacaaatactggttaattagcartttaagaccagagccaaattatccc

SEQ ID NO: 4 - DNA, 51 bp, Homo sapiens
atatacctgtcccactccactccccsacgcggcaaaccagcccttttatgg

(19) United States
(12) Patent Application Publication
(10) Pub. No.: US 2014/0288121 A1
(43) Pub. Date: Sep. 25, 2014

(54) Asthma

(71) Applicants: THE UNIVERSITY OF BRIGHTON, Brighton (GB); THE UNIVERSITY OF SUSSEX, Brighton (GB)

(72) Inventors: Somnath Mukhopadhyay, Brighton (GB); Jason Cunningham, Brighton (GB)

(21) Appl. No.: 14/214,286

(22) Filed: Mar. 14, 2014

Related U.S. Application Data
(60) Provisional application No. 61/789,437, filed on Mar. 15, 2013.

(51) Int. Cl. C12Q 1/68; A61K 31/4545

(52) U.S. Cl. CPC C12Q 1/6827; A61K 31/4545
USPC 514/318; 435/6.11

(57) ABSTRACT

The invention provides methods of providing a prognosis of asthma severity in children and young adults. More specifically, the invention relates to novel single nucleotide polymorphisms (SNPs), and the cumulative genetic association of these and other SNPs in various genes, with the prognosis of asthma severity. The invention also extends to kits and other prognostic tools used to provide the prognosis of asthma severity.

### CROSS-REFERENCE TO RELATED APPLICATION

The present application is a non-provisional of U.S. 61/789,437; filed Mar. 15, 2013, which is incorporated by reference its entirety for all purposes.

### FIELD OF THE INVENTION

The present invention relates to asthma, and in particular to methods of providing a prognosis of asthma severity in children and young adults. More specifically, the invention relates to novel single nucleotide polymorphisms (SNPs), and the cumulative genetic association of these and other SNPs in various genes, with the prognosis of asthma severity. The invention also extends to kits and other prognostic tools used to provide the prognosis of asthma severity.

### BACKGROUND

Asthma is one of the leading causes of morbidity in children. It affects 1.1 million children in the UK and nearly 6.7 million (more than 1 in 20) children in the US. Asthma costs the UK approximately £996 million per year, and the US approximately $37.2 billion per year. Approximately 48% of this cost is attributed to hospital admission alone. It is thus useful to know if a child diagnosed with asthma will remain asthmatic throughout their life, or if they may grow out of it as they get older. In addition, it would also be useful to know whether or not a child's asthma will remain mild throughout their life causing only minor problems, or if it will progress into the ‘severe’ form of the disease, which would necessitate hospital admissions, school absences, and long-term dependence on multiple medicines. Predicting the child's course of asthma (i.e. mild vs severe) would lead to the focusing of clinician's attention on children at a greater risk of developing more severe disease. This may include a closer monitoring of symptoms and peak flows at home, and earlier, more robust management with asthma medicines, etc. Such a strategy, focused on a smaller proportion of children and young adults, would save health services considerable resources, as clinicians could target only about 5-10% of children with asthma for more intensive follow-up. There are also major advantages to the identification of children with a greater risk of hospital admissions, emergency care visits, and a greater long-term need for asthma medication, for insurance agencies, in countries with private healthcare, as the accurate prediction of risk of disease severity usually forms an important part of their business strategy.

Identification of children and young adults at increased risk of severe asthma could reduce the healthcare costs of children and young adults with asthma. Clinical trials of drugs that target the underlying genetic susceptibilities in sub-populations of young asthmatics may lead to the development of novel interventions that could alter the development or extent of severe asthma in young people. Studies have identified individual candidate gene variants that are associated with a more severe asthma phenotype. However, such variants have limited use in the prediction of disease severity, since most of them confer only a relatively small risk.

### SUMMARY OF THE CLAIMED INVENTION

The invention provides methods for providing a prognosis for an individual's asthma severity, the method comprising detecting, in a sample obtained from the individual, for the presence of a genetic polymorphism pattern in a matrix metalloproteinase 9 (MMP9) gene, wherein the polymorphism pattern is associated with asthma severity. Optionally, the polymorphism pattern in MMP9 comprises a MMP9 rs17576 SNP. Optionally, the method comprises detecting for the presence of the A-allele of the MMP9 rs17576 SNP. Optionally, the polymorphism pattern in MMP9 comprises a MMP9 rs6073983 SNP. Optionally, the method comprises detecting for the presence of the A-allele of the MMP9 rs6073983 SNP. Optionally, the method comprises detecting, in the sample, for the presence of a genetic polymorphism pattern in a matrix metalloproteinase 12 (MMP12) gene and/or a Chitinase 3-like 1 (CH13L1) gene, wherein the polymorphism pattern is associated with asthma severity. Optionally, the method comprises detecting for the presence of the G-allele of the MMP12 rs652438 SNP. Optionally, the method comprises detecting for the presence of the C-allele of the CHI3L1 rs4950928 SNP. Optionally, the method comprises detecting two, three or four genetic polymorphism patterns in MMP9, MMP12 or CH13L1.

The invention further provides a method for providing a prognosis for an individual's asthma severity, the method comprising detecting, in a sample obtained from the individual, for the presence of a genetic polymorphism pattern in at least two genes selected from the group consisting of: matrix metalloproteinase 9 (MMP9); matrix metalloproteinase 12 (MMP12) gene; and Chitinase 3-like 1 (CH13L1) gene, wherein the polymorphism pattern is associated with asthma severity. Optionally, the method comprises detecting, in the sample, for the presence of a genetic polymorphism pattern in MMP9; MMP12; and/or CH13L1, wherein the polymorphism pattern is associated with asthma severity. Optionally, the method comprises detecting for the G-allele the MMP12 rs652438 SNP, the C-allele of the CHI3L1 rs4950928 SNP, and either the A-allele of the MMP9 rs17576 SNP or the A-allele of the MMP9 rs6073983 SNP, or both. Optionally the sample comprises a biological sample selected from blood, plasma, serum, spinal fluid, urine, sweat, saliva, tears, breast aspirate, prostate fluid, seminal fluid, vaginal fluid, stool, cervical scraping, cytes, amniotic fluid, intraocular fluid, mucous, moisture in breath, animal tissue, cell lysates, tumor tissue, hair, skin, buccal scrapings, nails, bone marrow, cartilage, prions, bone powder, ear wax, or combinations thereof. Optionally, the detecting step comprises use of at least one oligonucleotide operable to be used for amplification of genomic DNA encoding MMP9, MMP12 and/or CH13L1. Optionally, the oligonucleotide comprises a sequence selected from the group consisting of: SEQ ID No's 1-4, or a functional fragment or variant thereof. The invention further provides an asthma prognostic kit for identifying an individual's genetic polymorphism pattern in a matrix metalloproteinase 9 (MMP9) gene, wherein the polymorphism pattern is associated with asthma severity, the kit comprising means for determining the presence of a genetic polymorphism pattern in an MMP9 gene.

The invention further provides an asthma prognostic kit for identifying an individual's genetic polymorphism pattern in at least two genes selected from the group consisting of: a matrix metalloproteinase 9 (MMP9) gene; a matrix metalloproteinase 12 (MMP12) gene; and a Chitinase 3-like 1 (CH13L1) gene, wherein the polymorphism pattern is associated with asthma severity, the kit comprising means for determining the presence of at least two genetic polymorphism patterns in genes selected from the group consisting of: MMP9; MMP12; and CH13L1. Optionally, the kit comprises means for detecting for: (i) the G-allele the MMP12 rs652438 SNP, (ii) the C-allele of the CHI3L1 rs4950928 SNP, (iii) or the A-allele of the MMP9 rs17576 SNP, or (iv) the A-allele of the MMP9 rs6073983 SNP.

The invention further provides a method of treating an individual having a susceptibility to developing severe asthma, the method comprising:
(i) determining the genotype of an individual to identify the presence of a polymorphism pattern in a matrix metalloproteinase 9 (MMP9) gene, which polymorphism pattern is correlated with asthma severity; and
(ii) administering, to the individual, a therapeutic agent that prevents, reduces or delays progression of asthma.

The invention further provides a method of identifying an allele which is associated with asthma severity, the method comprising identifying an allele which is in linkage disequilibrium with the MMP12 rs652438 SNP and/or CHI3L1 rs4950928 SNP and/or MMP9 rs17576 SNP and/or MMP9 rs6073983 SNP, wherein said SNP is associated with asthma severity.

### BRIEF DESCRIPTION OF THE DRAWINGS

For a better understanding of the invention, and to show how embodiments of the same may be carried into effect, reference will now be made, by way of example, to the accompanying Figures, in which:

FIG. 1 summarises the role of the molecules within the overall process of airway remodeling;

FIG. 2 is an illustration of the overall cumulative effect of the CHI3L1 rs4950928, MMP9 SNP rs6073983, MMP9 SNP rs17576, MMP12 rs652438 genotypes (risk and protective variants) on asthma exacerbations in children and young adults recruited to the BREATHE study;

FIG. 3 is an illustration of the overall cumulative effect of the CHI3L1 rs4950928, MMP9 SNP rs6073983, MMP9 SNP rs17576, MMP12 rs652438 genotypes (risk and protective variants) on asthma severity measured by CAASS in children and young adults recruited to the BREATHE study. Count (%), odds ratio (95% confidence intervals), P values calculated by binary or multinomial logistic regression, adjusted for age & sex;

FIGS. 4A and 4B show associations of CHI3L1 rs4950928, MMP9 rs17576, MMP9 rs6073983, MMP12 rs652438 with measures of asthma severity; and

FIGS. 5A and 5B show the cumulative effect of number of risk variants CHI3L1 rs4950928, MMP9 rs17576, MMP9 rs6073983 and MMP12 rs652438 on measures of asthma severity. Count (%), odds ratio (95% confidence intervals), P values calculated by binary or multinomial logistic regression adjusted for age and sex.

### DETAILED DESCRIPTION

There is therefore a need to provide a more reliable and accurate means for providing a prognosis of the severity of asthma.

According to a first aspect of the invention, there is provided a method for providing a prognosis for an individual's asthma severity, the method comprising detecting, in a sample obtained from the individual, for the presence of a genetic polymorphism pattern in a matrix metalloproteinase 9 (MMP9) gene, wherein the polymorphism pattern is associated with asthma severity.

It will be appreciated that the method of the first aspect does not relate to determining whether or not the individual will develop asthma per se. Instead, it involves the prediction of how severe (or mild) the asthma will be after the individual has developed the disease. Advantageously, the method of the invention enables the prediction of an individual (for example, a child) who will have relatively mild asthma as they get older, compared to an individual who will progress to developing more severe asthma. The invention may also be used to identify individuals who are at a greater risk of severe airway remodeling and who could benefit from the early introduction of more intensive therapy with asthma treatments, including the use of inflammation-blocking treatments, and targeting community healthcare interventions (such as greater primary care input, asthma management plans, more careful discharge planning etc). Also, the method of the invention enables the use of targeted therapies with novel medications, such as MMP9 or MMP12 inhibitors. To date, it has not been possible to achieve any of these advantages using known asthma diagnostics.

The MMP9 gene is located on chromosome 20. The coding DNA sequence of MMP9 is known and is readily accessible at www.ncbi.nlm.nih.gov. The polymorphism pattern in MMP9, which is detected for in the method of the invention, may comprise at least one polymorphism or polymorphic region of the MMP9 gene.

The term “polymorphism” can refer to the co-existence, within a population, of more than one form of a gene or portion thereof (e.g. an allelic variant). A portion of a gene of which there are at least two different forms, i.e. two different nucleotide sequences, is referred to as a “polymorphic region of a gene”. A specific genetic sequence at a polymorphic region of a gene is known as an allele. The term “allele” can refer to the different sequence variants found at different polymorphic sites in DNA obtained from a subject. For example, each polymorphic region of the MMP9 gene has at least two different alleles. The sequence variants of each allele may be single or multiple base changes, including without limitation insertions, deletions, or substitutions, or may be a variable number of sequence repeats. Thus, a polymorphic region may be a single nucleotide (i.e. a single nucleotide polymorphism, or SNP), the identity of which differs in different alleles. A polymorphic region can also be several nucleotides long.

Preferably, the polymorphism pattern in MMP9 comprises more than one, and preferably two, polymorphic regions of the MMP9 gene. For example, in one embodiment, the polymorphism pattern in MMP9 may comprise a MMP9 rs17576 SNP. The alleles of the MMP9 rs17576 SNP may be identified as (i) an A-allele, and (ii) a G-allele. The inventors have surprisingly found that the A-allele of the MMP9 rs17576 SNP is associated with asthma severity, particularly in children. Hence, the method may comprise detecting for the presence of the A-allele of the MMP9 rs17576 SNP.

In another embodiment, the polymorphism pattern in MMP9 may comprise a MMP9 rs6073983 SNP. The alleles of the MMP9 rs6073983 SNP may be identified as (i) an A-allele, and (ii) a T-allele. The inventors have found that the A-allele of the MMP9 rs6073983 SNP is associated with asthma severity, particularly in children. Hence, the method may comprise detecting for the presence of the A-allele of the MMP9 rs6073983 SNP.

Preferably, the method of the invention comprises detecting, in the sample, for the presence of the two MMP9 SNPs. The inventors have found surprisingly strong association of the two MMP9 SNPs with severe asthma. As described in the Examples, the inventors also investigated other SNPs involved in asthma prognosis (i.e. the MMP12 and CH13L1 genes).

Accordingly, the method may further comprise detecting, in the sample, for the presence of a genetic polymorphism pattern in a matrix metalloproteinase 12 (MMP12) gene and/or a Chitinase 3-like 1 (CH13L1) gene, wherein the polymorphism pattern is associated with asthma severity.

The inventors were very surprised to observe that the relationship between the number of polymorphisms that an individual has and the severity of their asthma is additive (i.e. cumulative). In other words, an individual with two of these SNPs developed more severe asthma than an individual having just one SNP, and the asthma in an individual having three SNPs was more severe than in an individual having just two SNPs. The results also show that an individual who has all four SNPs developed asthma that was more severe than an individual with three of the SNPs. The inventors were surprised at such a finding, as it was unexpected that an individual having all four SNPs described herein would have more severe asthma than an individual having three, two, or only one of the SNPs.

The MMP12 gene is located on chromosome ii. The polymorphism pattern in MMP12 may comprise at least one polymorphism or polymorphic region of the MMP12 gene. In one embodiment, the polymorphism pattern in MMP12 may comprise a MMP12 rs652438 SNP. The alleles of the MMP12 rs652438 SNP may be identified as (i) an A-allele, and (ii) a G-allele. The inventors have confirmed previous observations that the G-allele of the MMP12 rs652438 SNP is associated with asthma severity, particularly in children. Hence, the method may comprise detecting for the presence of the G-allele in the MMP12 rs652438 SNP.

The Chitinase 3-like 1 (CH13L1) gene is located on chromosome 1. The polymorphism pattern in CH13L may comprise at least one polymorphism or polymorphic region of the CH13L gene. In one embodiment, the polymorphism pattern in CH13L1 may comprise a CH13L1 rs4950928 SNP. The alleles of the CH13L1 rs4950928 SNP may be identified as (i) a C-allele, and (ii) a G-allele. The inventors have confirmed previous observations that the C-allele of the CHI3L1 rs4950928 SNP is associated with asthma severity, particularly in children. Hence, the method may comprise detecting for the presence of the C-allele the CH13L1 rs4950928 SNP.

It will be appreciated therefore that the method of the invention may comprise detecting for the presence of either or both of the MMP9 SNP's and the MMP12 SNP and/or the CH13L1 SNP.

The method may therefore comprise detecting two or more of the genetic polymorphisms in MMP9, MMP12 or CH13L1, and particularly, three or more of the alleles described herein as being associated with severe asthma. Preferably, and advantageously, prognosis of the severity of asthma may be carried out by detection of four of the polymorphic regions, as described herein. Preferably, prognosis of asthma severity may be carried out by detecting which alleles of the various polymorphic regions are present. Advantageously, screening for the presence of two or more of the alleles associated with asthma severity allows for the identification of individuals likely to have a genetic susceptibility to developing more severe forms of asthma.

However, the inventors have realised that this cumulative effect means that, in some embodiments, it may not be desirable to detect for the presence of the MMP9 SNPs, and it may be possible to detect for the presence of only the MMP12 and CH13L1 SNPs.

Therefore, according to a second aspect of the invention, there is provided a method for providing a prognosis for an individual's asthma severity, the method comprising detecting, in a sample obtained from the individual, for the presence of a genetic polymorphism pattern in at least two genes selected from the group consisting of: matrix metalloproteinase 9 (MMP9); metalloproteinase 12 (MMP12) gene; and Chitinase 3-like 1 (CH13L1) gene, wherein the polymorphism pattern is associated with asthma severity.

The inventors have found strong association of the two MMP9 SNPs with severe asthma. Accordingly, in one embodiment, the method may comprise detecting for the presence of a genetic polymorphism in MMP9 only. In this embodiment, the method may not comprise detecting for the presence of a genetic polymorphism MMP12 and/or CH13L1.

Preferably, in carrying out the method of the invention, an individual's genotype is determined by analysis of a region of the MMP9, MMP12 and/or CH13L1 gene, rather than by analysis of each entire gene sequence. The term “genotype”, “allelic pattern” or “polymorphism pattern” can refer to the identity of an allele or alleles at one or more polymorphic sites. A genotype, allelic pattern or polymorphism pattern may consist of either a homozygous or heterozygous state at one or more polymorphic sites.

Preferably, in an embodiment of the invention, the susceptibility to developing severe asthma is assessed by determining whether an individual has any of the “high risk” alleles (i.e. those alleles associated with severe asthma). According to the invention, an individual who carries two or more of the high risk alleles (i.e. the alleles associated with severe asthma) is classified as being at a higher risk for developing severe asthma when compared to an individual who has none of the high risk alleles (i.e. the other of the two alleles) for the polymorphic regions. Asthma severity may be defined as the occurrence of an exacerbation in the last 6 months (hospital admission, oral steroid use or school absence) and/or requirement of increased levels of regular asthma medication (described as higher asthma drug class or higher asthma treatment step), adjusted by bronchodilator use and the occurrence of an exacerbation.

Therefore, the method may comprise detecting, in the sample, for the presence of a genetic polymorphism pattern in at least three genes selected from the group consisting of: MMP9; MMP12; and CH13L1, wherein the polymorphism pattern is associated with asthma severity.

For example, in one embodiment, the method of the first or second aspect may comprise detecting for the presence of a genetic polymorphism pattern in MMP9 and MMP12. In this embodiment, the method may comprise detecting for the G-allele of the MMP12 rs652438 SNP and the A-allele of the MMP9 rs17576 SNP and/or the A-allele of the MMP9 rs6073983 SNP.

In another embodiment, the method may comprise detecting for the presence of a genetic polymorphism pattern in MMP9 and CH13L. In this embodiment, the method may comprise detecting for the C-allele the CHI3L1 rs4950928 SNP and the A-allele of the MMP9 rs17576 SNP and/or the A-allele the MMP9 rs6073983 SNP.

In yet another embodiment, the method may comprise detecting for the presence of a genetic polymorphism pattern in MMP12 and CH13L1. In this embodiment, the method may comprise detecting for the G-allele the MMP12 rs652438 SNP and the C-allele the CHI3L1 rs4950928 SNP.

However, preferably the methods of the invention comprise detecting for the presence of a genetic polymorphism pattern in all three genes, i.e. in MMP9, MMP12 and CH13L1 genes. In this embodiment, the method may comprise detecting for the G-allele the MMP12 rs652438 SNP, the C-allele of the CH13L1 rs4950928 SNP, and either the A-allele of the MMP9 rs17576 SNP or the A-allele of the MMP9 rs6073983 SNP, or both.

The polymorphism pattern may comprise a further polymorphism which is in linkage disequilibrium with at least one polymorphism which is known to be associated with asthma severity. In a preferred embodiment, any two of the polymorphic regions correlate with asthma severity. Preferably, at least two of said polymorphisms are in linkage disequilibrium with each other.

“Linkage disequilibrium” refers to the co-inheritance of two or more alleles at frequencies greater than would be expected from the separate frequencies of occurrence of each allele in a given control population. The expected frequency of occurrence of two alleles that are inherited independently is the frequency of the first allele in that population multiplied by the frequency of the second allele in that population. Alleles that co-occur at expected frequencies are said to be in “linkage equilibrium”. The cause of linkage disequilibrium is often unclear. It can be due to selection for certain allele combinations or to recent admixture of genetically heterogeneous populations. In addition, in the case of genetic markers that are very tightly linked to a disease gene, an association of an allele (or group of linked alleles) with the disease gene is expected if the disease mutation occurred in the recent past, so that sufficient time has not elapsed for equilibrium to be achieved through recombination events in the specific chromosomal region. When referring to allelic patterns or polymorphism patterns that comprise more than one allele, a first allelic pattern is in linkage disequilibrium with a second allelic pattern if at least one of the alleles that comprise the first allelic pattern is in linkage disequilibrium with at least one of the alleles of the second allelic pattern.

Preferably the sample comprises a biological sample. The sample may be any material that is obtainable from a subject from which genomic DNA or cDNA is obtainable. Furthermore, a sample may be blood, plasma, serum, spinal fluid, urine, sweat, saliva, tears, breast aspirate, prostate fluid, seminal fluid, vaginal fluid, stool, cervical scraping, cytes, amniotic fluid, intraocular fluid, mucous, moisture in breath, animal tissue, cell lysates, tumor tissue, hair, skin, buccal scrapings, nails, bone marrow, cartilage, prions, bone powder, ear wax, or combinations thereof.

The term “detecting alleles” refers to the process of genotyping, genetic testing, genetic screening, determining or identifying an allele or polymorphism. The allele actually detected may be a disease-associated polymorphic allele, or a mutation that is in linkage disequilibrium with such an allele. It will manifest in the genomic DNA of an individual, but may also be detectable from RNA or protein sequences transcribed or translated from that region. Accordingly, preferably the sample comprises nucleic acid. The nucleic acid tested may be RNA or DNA, although DNA is preferred. Preferably, the sample comprises genomic DNA. Preferably, the nucleic acid encodes at the polymorphic region of MMP9, MMP12 and/or CH13L1.

Techniques for determining the presence of particular alleles are known the skilled person and include, but are not limited to, nucleic acid techniques based on size or sequence, such as restriction fragment length polymorphism (RFLP), nucleic acid sequencing, or nucleic acid hybridization. These techniques may also comprise the step of amplifying the nucleic acid before analysis. Amplification techniques are known to the skilled person and may include, but are not limited to, cloning, polymerase chain reaction (PCR), polymerase chain reaction of specific alleles (PASA), polymerase chain ligation, nested polymerase chain reaction, and the like.

Thus, the detecting step may comprise amplification of the sample, for example PCR amplification. PCR involves amplifying DNA, preferably small amounts of DNA, to ease subsequent detection of the genetic polymorphic patterns. Many variations of the basic amplification protocol are well-known to those of skill in the art. PCR-based detection means include multiplex amplification of a plurality of polymorphisms or markers, simultaneously. For example, it is well known to select PCR primers to generate PCR products that do not overlap in size and which can be analysed simultaneously. Alternatively, it is possible to amplify different markers with primers that are differentially labelled and thus can each be differentially detected. Of course, hybridization-based detection means allow the differential detection of multiple PCR products in a sample. Other techniques are known to allow multiplex analysis of a plurality of markers.

The detecting step may comprise conducting a genotyping assay, for example that which is available under the trade name Taqman SNP genotyping assay (Applied Biosystems Europe, Warrington, UK). TaqMan SNP genotyping assays consist of two context sequence amplifying primers and two allele specific TaqMan Minor Groove Binder (MGB) probes. Each allele specific probe preferably contains a reporter dye at the 5′ end. For example, the reporter dye for allele 1 may be VIC®, and the reporter dye for allele 2 may be FAM (6-carboxy-fluorescein). At the 3′ end of each allele specific probe there may be a non-fluorescent quencher, which serves to increase the accuracy of allelic discrimination by suppressing fluorescence from the reporter dye, whilst the probe is intact. During the assay, each probe anneals to a specific complimentary sequence between the forward and reverse primer sites. Hybridized probes are cleaved by AmpliTaq Gold® polymerase, separating the reporter dye from the quencher dye. Incomplete hybridization, or mismatches between the probe and the target, are dislodged by AmpliTaq Gold® polymerase without cleaving the probe.

Hence, preferably the detecting step comprises use of at least one oligonucleotide operable to be used for amplification of genomic DNA encoding MMP9, MMP12 and/or CH13L1. Accordingly, genotyping for missense A/G transition substitution MMP9 rs17576 may be performed using a Taqman SNP genotyping assay c_{—}11655953_{—}10 (Applied Biosystems Europe, Warrington, UK). The primer sequence used may be: 5′-CTCCTCGCCCCAGGACTCTACACCC[A/G]GGACGGCAATGCTGATGGGAAACCC-3′ [SEQ ID No:2]. The A allele may be tagged with VIC® dye, and the G allele was tagged with FAM® dye.

Genotyping for the promoter A/T transversion substitution MMP9 rs6073983 may be performed using a Taqman SNP genotyping assay c_{—}30627092_{—}10 (Applied Biosystems Europe, Warrington, UK). The primer sequence used may be: 5′-AGGTGAAAGTCAGGCATCAGTATTC[A/T]AGAGAAAATCCTGAGTGATTCCAAT-3′ [SEQ ID No:2]. The A allele may be tagged with VIC® and the T allele may be tagged with FAM®.

Genotyping for the coding C/T transition substitution MMP12 rs652438 may be performed using a Taqman SNP genotyping assay c_{—}785907_{—}10 (Applied Biosystems Europe, Warrington, UK). The primer sequence may be: 5′-AGATGACAAATACTGGTTAATTAGCA[A/G]TTTAAGACCAGAGCCAAATTATCCC-3′ [SEQ ID No:3]. The C allele may be tagged with VIC® and the T allele may be tagged with FAM®.

Genotyping for the functional promoter C/G transversion substitution CHI3L1 rs4950928 was performed using a Taqman SNP genotyping assay c_{—}27832042_{—}10 (Applied Biosystems Europe, Warrington, UK). The primer sequence may be: 5′-ATATACCTGTCCCACTCCACTCCCC[C/G]ACGCGGCAAACCAGCCCTITTATGG-3′ [SEQ ID No:4]. The C allele was tagged with VIC® and the G allele was tagged with FAM®.

Preferably, the PCR amplification employs at least one primer comprising a sequence selected from the group consisting of: SEQ ID No's 1-4, or a functional fragment or variant thereof.

Amplification products may be assayed in a variety of ways, including size analysis, restriction digestion followed by size analysis, detecting specific tagged oligonucleotide primers in the reaction products, allele specific oligonucleotide (ASO) hybridization, allele specific S′ exonuclease detection, sequencing, hybridization and the like. Polymorphic variations leading to altered protein sequences or structures may also be detected by analysis of the protein itself. Preferably, said detecting comprises subjecting the amplified DNA to size analysis, for example electrophoresis and, preferably comparing the results to a positive control and, preferably a negative control.

Said size analysis may be preceded by restriction enzyme digestion. Said detecting may comprise digesting the amplified DNA with a restriction enzyme, and then, preferably, subjecting the resultant digested DNA to electrophoresis and, preferably, comparing the results to a positive and, preferably, a negative control.

Alternatively, or additionally, the gel may undergo Southern blotting or other hybridization analyses comprising labeling, preferably radio-labeling a probe. Said detecting may comprise sequencing the DNA encoding the polymorphisms to determine the allele or alleles present.

The individual may be a vertebrate, mammal, or domestic animal. Most preferably, however, the individual is a human being. The individual may be a child or adult. For example, the individual may be less than 20 years old or less than 15 years old. The individual may be less than 10, 5, 3 or 2 years old. The individual may be a baby, i.e. 12 months old, or less, including a foetus. Preferably, the method is carried out in vitro.

The inventors have developed a kit which can be used to provide a prognosis for an individual's asthma severity.

Hence, according to a third aspect, there is provided an asthma prognostic kit for identifying an individual's genetic polymorphism pattern in a matrix metalloproteinase 9 (MMP9) gene, wherein the polymorphism pattern is associated with asthma severity, the kit comprising means for determining the presence of a genetic polymorphism pattern in an MMP9 gene.

The kit may comprise means for determining the presence of a genetic polymorphism pattern in a MMP12 gene and/or a CH13L1 gene.

Thus, according to a fourth aspect, there is provided an asthma prognostic kit for identifying an individual's genetic polymorphism pattern in at least two genes selected from the group consisting of: a matrix metalloproteinase 9 (MMP9) gene; a matrix metalloproteinase 12 (MMP12) gene; and a Chitinase 3-like 1 (CH13L1) gene, wherein the polymorphism pattern is associated with asthma severity, the kit comprising means for determining the presence of at least two genetic polymorphism patterns in genes selected from the group consisting of: MMP9; MMP12; and CH13L1.

Preferably, the kits of the invention are used to provide a prognosis for an individual's asthma severity. The kit may be used to identify individuals who will develop mild or severe forms of asthma. The means for determining the presence of the polymorphism pattern may comprise analysis of the DNA sample, more preferably genetic analysis.

It will be appreciated that the inventors have determined that there are four SNPs which are associated with asthma severity, and the kits of the invention may comprise means for detecting any or all of these SNPs. The kit may therefore comprise means for detecting for: (i) the G-allele the MMP12 rs652438 SNP, (ii) the C-allele of the CHI3L1 rs4950928 SNP, (iii) or the A-allele of the MMP9 rs17576 SNP, or (iv) the A-allele of the MMP9 rs6073983 SNP. However, as described herein, there is a cumulative effect in that the more SNPs an individual has, the more severe their asthma will be in later life.

Thus, the kit may comprise means for determining the presence of a genetic polymorphism pattern in at least two genes selected from the group consisting of: MMP9; MMP12; and CH13L. For example, the kit may comprise means for determining the presence of a genetic polymorphism pattern in MMP9 and MMP12, or in MMP9 and CH13L, or MMP12 and CH13L.

However, preferably the kit comprises means for determining the presence of a genetic polymorphism pattern in the MMP9, MMP12 and CH13L genes (i.e. all three genes). In this embodiment, the kit may comprise means for detecting for the G-allele the MMP12 rs652438 SNP, the C-allele of the CHI3L1 rs4950928 SNP, and either the A-allele of the MMP9 rs17576 SNP or the A-allele of the MMP9 rs6073983 SNP, or both.

Preferably, the kit comprises DNA sampling reagents and, preferably PCR amplification reagents. Preferably, the PCR amplification reagents comprise the use of Ampli-Taq Gold.

Oligonucleotide DNA primers that target the specific polymorphism DNA region within the genes of interest may be prepared so that, in the PCR reaction, amplification of the target sequences may be achieved.

In one embodiment, the kit may comprise c_{—}11655953_{—}10 (SEQ ID No:1, for genotyping MMP9 rs17576).

In another embodiment, the kit may comprise c_{—}30627092_{—}10 (SEQ ID No:2, for genotyping MMP9 rs6073983).

In another embodiment, the kit may comprise c_{—}785907_{—}10 (SEQ ID No:3, for genotyping MMP12 rs652438).

In yet another embodiment, the kit may comprise c_{—}27832042_{—}10 (SEQ ID No:4, for genotyping CHI3L1 rs4950928).

Thus, the kit may comprise at least one primer comprising a sequence selected from the group consisting of: SEQ ID No's 1-4, or a functional fragment or variant thereof. Said primers may comprise one or more detectable label, for example FAM® or VIC®.

Preferably, the kit comprises at least one control sample. The kit may comprise a negative control and/or a positive control. A positive control may comprise one or more of the alleles known to be associated with severe asthma. A negative control may comprise one or more of the other alleles which is not associated with severe asthma. Preferably, the kit comprises means to compare the polymorphism pattern to a control sample of known asthma severity to provide a prognosis for an individual's asthma severity.

The invention extends to methods of treatment.

Hence, according to a fifth aspect of the invention, there is provided a method of treating an individual having a susceptibility to developing severe asthma, the method comprising:
(i) determining the genotype of an individual to identify the presence of a polymorphism pattern in a matrix metalloproteinase 9 (MMP9) gene, which polymorphism pattern is correlated with asthma severity; and
(ii) administering, to the individual, a therapeutic agent that prevents, reduces or delays progression of asthma.

The polymorphism pattern may be in at least two genes selected from the group consisting of: MMP9; MMP12; and CH13L1.

The invention also extends to identifying novel SNPs which are associated with asthma severity.

Hence, according to a sixth aspect, there is provided a method of identifying an allele which is associated with asthma severity, the method comprising identifying an allele which is in linkage disequilibrium with the MMP12 rs652438 SNP and/or CHI3L1 rs4950928 SNP and/or MMP9 rs17576 SNP and/or MMP9 rs6073983 SNP, wherein said SNP is associated with asthma severity.

The skilled person will appreciate how to conduct linkage disequilibrium analysis based on one of more of the SNPs described herein, in order to identify a new allele or SNP which can also be used a prognostic marker for severe asthma.

It will be appreciated that the invention extends to any nucleic acid or peptide or variant, derivative or analogue thereof, which comprises substantially the amino acid or nucleic acid sequences of any of the sequences referred to herein, including functional variants or functional fragments thereof. The terms “substantially the amino acid/nucleotide/peptide sequence”, “functional variant” and “functional fragment”, can be a sequence that has at least 40% sequence identity with the amino acid/nucleotide/peptide sequences of any one of the sequences referred to herein, for example 40% identity with the nucleotide identified as SEQ ID No:1-4.

Amino acid/polynucleotide/polypeptide sequences with a sequence identity which is greater than 50%, more preferably greater than 65%, 70%, 75%, and still more preferably greater than 80% sequence identity to any of the sequences referred to are also envisaged. Preferably, the amino acid/polynucleotide/polypeptide sequence has at least 85% identity with any of the sequences referred to, more preferably at least 90%, 92%, 95%, 97%, 98%, and most preferably at least 99% identity with any of the sequences referred to herein.

The skilled technician will appreciate how to calculate the percentage identity between two amino acid/polynucleotide/polypeptide sequences. In order to calculate the percentage identity between two amino acid/polynucleotide/polypeptide sequences, an alignment of the two sequences must first be prepared, followed by calculation of the sequence identity value. The percentage identity for two sequences may take different values depending on:—(i) the method used to align the sequences, for example, ClustalW, BLAST, FASTA, Smith-Waterman (implemented in different programs), or structural alignment from 3D comparison; and (ii) the parameters used by the alignment method, for example, local vs global alignment, the pair-score matrix used (e.g. BLOSUM62, PAM250, Gonnet etc.), and gap-penalty, e.g. functional form and constants.

Having made the alignment, there are many different ways of calculating percentage identity between the two sequences. For example, one may divide the number of identities by: (i) the length of shortest sequence; (ii) the length of alignment; (iii) the mean length of sequence; (iv) the number of non-gap positions; or (iv) the number of equivalenced positions excluding overhangs. Furthermore, it will be appreciated that percentage identity is also strongly length dependent. Therefore, the shorter a pair of sequences is, the higher the sequence identity one may expect to occur by chance.

Hence, it will be appreciated that the accurate alignment of protein or DNA sequences is a complex process. The popular multiple alignment program ClustalW (Thompson et al., 1994, Nucleic Acids Research, 22, 4673-4680; Thompson et al., 1997, Nucleic Acids Research, 24, 4876-4882) is a preferred way for generating multiple alignments of proteins or DNA in accordance with the invention. Suitable parameters for ClustalW may be as follows: For DNA alignments: Gap Open Penalty=15.0, Gap Extension Penalty=6.66, and Matrix=Identity. For protein alignments: Gap Open Penalty=10.0, Gap Extension Penalty=0.2, and Matrix=Gonnet. For DNA and Protein alignments: ENDGAP=−1, and GAPDIST=4. Those skilled in the art will be aware that it may be necessary to vary these and other parameters for optimal sequence alignment.

Preferably, calculation of percentage identities between two amino acid/polynucleotide/polypeptide sequences may then be calculated from such an alignment as (N/T)*100, where N is the number of positions at which the sequences share an identical residue, and T is the total number of positions compared including gaps but excluding overhangs. Hence, a most preferred method for calculating percentage identity between two sequences comprises (i) preparing a sequence alignment using the ClustalW program using a suitable set of parameters, for example, as set out above; and (ii) inserting the values of N and T into the following formula:—Sequence Identity=(N/T)*100.

Alternative methods for identifying similar sequences will be known to those skilled in the art. For example, a substantially similar nucleotide sequence will be encoded by a sequence which hybridizes to the sequences shown in SEQ ID No's:1-4, or their complements under stringent conditions. By stringent conditions, we mean the nucleotide hybridises to filter-bound DNA or RNA in 3× sodium chloride/sodium citrate (SSC) at approximately 45° C. followed by at least one wash in 0.2×SSC/0.1% SDS at approximately 20-65° C.

Due to the degeneracy of the genetic code, it is clear that any nucleic acid sequence described herein could be varied or changed without substantially affecting the sequence of the protein encoded thereby, to provide a functional variant thereof. Suitable nucleotide variants are those having a sequence altered by the substitution of different codons that encode the same amino acid within the sequence, thus producing a silent change. Other suitable variants are those having homologous nucleotide sequences but comprising all, or portions of, sequence, which are altered by the substitution of different codons that encode an amino acid with a side chain of similar biophysical properties to the amino acid it substitutes, to produce a conservative change. For example small non-polar, hydrophobic amino acids include glycine, alanine, leucine, isoleucine, valine, proline, and methionine. Large non-polar, hydrophobic amino acids include phenylalanine, tryptophan and tyrosine. The polar neutral amino acids include serine, threonine, cysteine, asparagine and glutamine. The positively charged (basic) amino acids include lysine, arginine and histidine. The negatively charged (acidic) amino acids include aspartic acid and glutamic acid. It will therefore be appreciated which amino acids may be replaced with an amino acid having similar biophysical properties, and the skilled technician will know the nucleotide sequences encoding these amino acids.

All of the features described herein (including any accompanying claims, abstract and drawings), and/or all of the steps of any method or process so disclosed, may be combined with any of the above aspects in any combination, except combinations where at least some of such features and/or steps are mutually exclusive.

### EXAMPLES

### Materials & Methods

### Participants

Study participants consisted of children and young adults with physician-diagnosed asthma (age 3-22 years). They were recruited from the BREATHE study, an investigation of individual gene-environment associations with asthma severity in the UK. The BREATHE study was approved by the Tayside Committee on Medical Research and Ethics. Information was obtained at the asthma clinic on school/college/work absences, use of oral steroids, asthma-related hospital admissions and medication use, over the previous 6 months. Genomic DNA was isolated from saliva samples from 1332 subjects after the patient and/or parent/guardian had provided written informed consent.

### Genotyping

Genotyping for all four variants was performed using the Taqman SNP genotyping assay (Applied Biosystems Europe, Warrington, UK). Genotyping for: (i) CH13L1 rs4950928 was performed using c_{—}27832042_{—}10, (ii) MMP9 rs17576 was performed using c_{—}11655953_{—}10, (iii) MMP9 rs6073983 was performed using c_{—}30627092_{—}10 and (iv) MMP12 rs652438 was performed using c_{—}785907_{—}10.

Amplification was performed in 10 μl wells. Each well contained allele specific primers and AmpliTaq Gold® PCR Master Mix (Applied Biosystems). Each well contains 2.25 μl of DNase free water diluted sample at a 1 to 20 ng dilution, 0.25 μl of primer mix, 2.5 μl of AmpliTaq Gold® PCR Master Mix, comprising 1.5mM MgCl2, 10 nmol of each dNTP and 1 unit AmpliTaq Gold DNA polymerase. Amplification was performed using thermal cyclers. Cycles consisted of three water baths, two of 95° C. and one at 65° C. Allelic discrimination assays required 60 cycles to amplify.

### Measures of Severity

A combined exacerbation score, involving yes/no responses for any of the three measures of exacerbations (asthma-related school/college/work absences, use of oral steroids, asthma-related hospital admissions) over a 6 month period of reporting was used. Asthma-related hospital admission, asthma-related school/college/work absences and oral steroid intake were grouped as present (minimum once over the previous 6 months) or absent. The total asthma exacerbation response was calculated as any of these measures during the same period of time and grouped as present or absent.

The asthma prescribing level or treatment step was modified from British Thoracic Society (BTS) guidelines (Bts/Sign. British guidelines on the management of asthma. Thorax 2003:1-94), as follows: step 1, inhaled short-acting beta-2 agonists on demand; step 2, regular inhaled steroids plus inhaled short-acting beta-2 agonists on demand; step 3a, regular inhaled long-acting beta-2 agonists (salmeterol or formoterol) plus inhaled steroids with inhaled short-acting beta-2 agonists on demand; step 4, regular inhaled long-acting beta-2 agonists plus inhaled steroids plus oral montelukast with inhaled short-acting beta-2 agonists on demand. The use of inhaled short-acting beta-2 agonists (bronchodilators) was categorized as not used (no use in the previous six months), occasional use (used occasionally over the previous 6 months), once daily use and more than once daily use.

From the measures described above, a global index of asthma severity was derived through construction of a composite variable in the form of inhaled bronchodilator use and asthma exacerbation adjusted asthma treatment step. This Childhood and Adolescent Asthma Severity Score (CAASS) ranges from 1-6, where 1 is equivalent to mild asthma and 6 corresponds to severe asthma. The score is derived from adjusting asthma treatment step by the frequency of bronchodilator use and/or the occurrence of an asthma exacerbation. Daily or more than daily use of inhaled bronchodilator over the previous 6 months, or the occurrence of an asthma exacerbation in the previous 6 months would increase the base line score (current asthma treatment step) by 1, or by 2 in the case of the occurrence of both events.

### Statistical Methods

A test for Hardy-Weinberg equilibrium was performed for each of the SNPs individually. Specimens that did not call for all 4 genotypes were excluded. Binary logistic regression was used to estimate the association between the individual gene variants and measures of asthma exacerbations and asthma severity. Multinomial logistic regression was used to estimate the associations between the gene variants and asthma treatment step and gene variants and CAASS. Odds ratios were adjusted for age and sex.

Strong biological dependence between the four risk variants allowed for a combined analysis in which the inventors were able to test the cumulative effect of these variants on asthma severity. For the purpose of investigating the cumulative effect of the investigated variants, the risk variants of each gene (CC variant of CH13L1 rs4950928; AA/AG variant of MMP9 rs17576; AA/AT variant of MMP9 rs6073983; AG/GG variant of MMP12 rs652438) were combined into a cumulative figure for each participant, ranging from o risk variants to 4 risk variants.

Data were analysed using SPSS version i8 for Windows (IBM, New York). All P values are two-tailed and values of less than 0.05 were considered to indicate statistical significance.

### Example 1

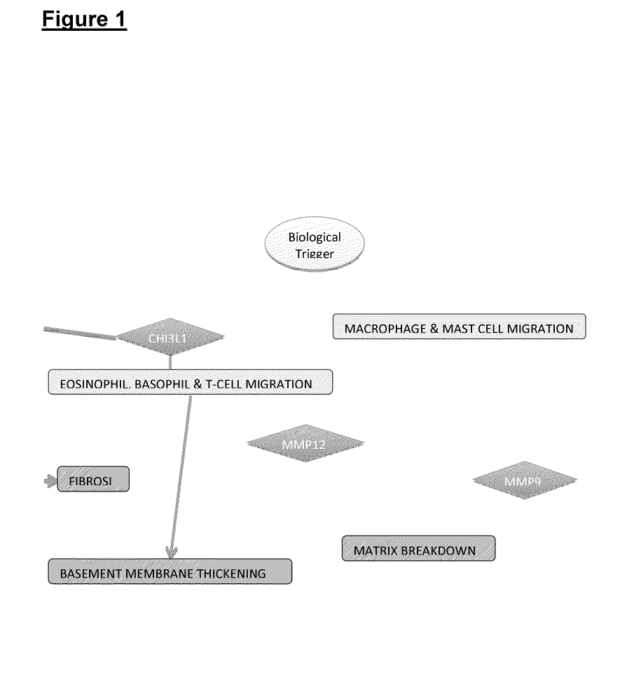

Four single nucleotide polymorphisms (SNPs) in three genes implicated in the airway remodeling process (see FIG. 1), were evaluated in 1332 young individuals (mean age 10.0 years (standard deviation 4.1 years), 786 (59%) male). Each SNP was in Hardy-Weinberg equilibrium (P≧0.05).

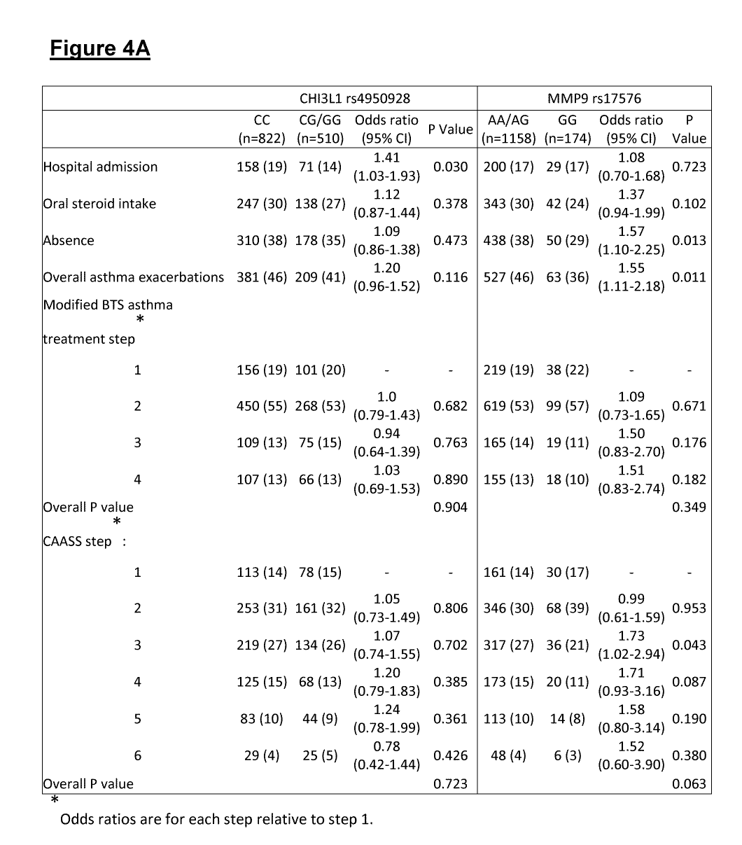

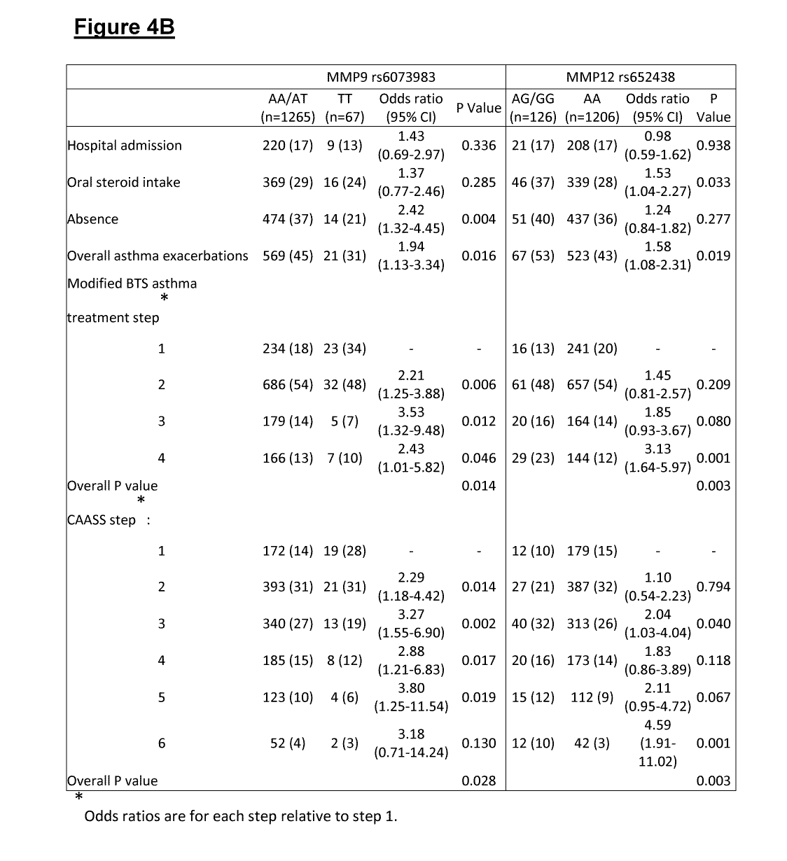

FIGS. 4A and 4B lists allele frequencies of the 4 SNPs and shows the individual SNP associations with asthma severity. Significant associations with asthma severity were observed for each of the SNPs. MMP12 SNP rs652438 had the strongest association with CAASS (odds ratio for CAASS level 6 compared with level 1=4.59; P=0.001). All the investigated SNPs were associated with at least one of asthma-related hospital admission, oral steroid use, absence from school, college or work, or any one of these (overall asthma exacerbations). MMP9 SNPs rs17576 and rs6073983 and MMP12 rs652439 were significantly associated with overall exacerbations. The strongest association for any measure of asthma exacerbation was between MMP9 SNP rs6073983 and school/college/work absence due to exacerbations (odds ratio=2.42; p=0.004). CHI3L1 SNP rs4950928 showed an association with hospital admissions (odds ratio=1.41; p=0.030).

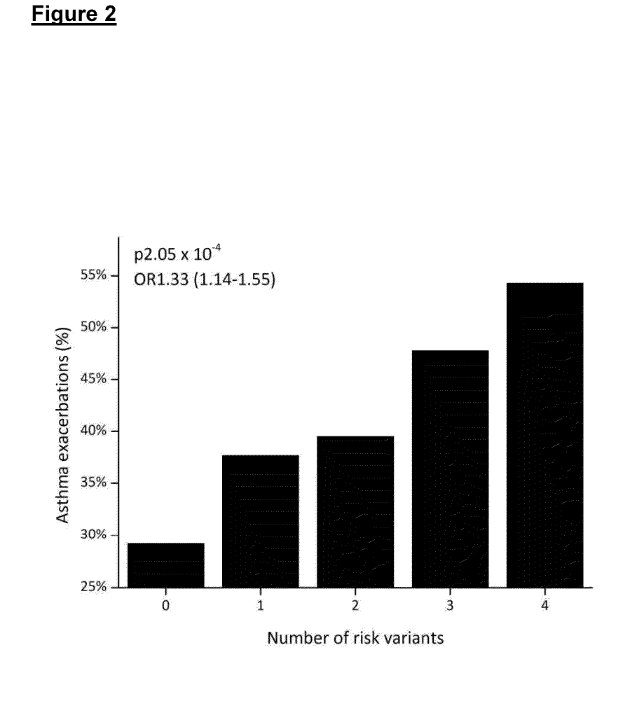

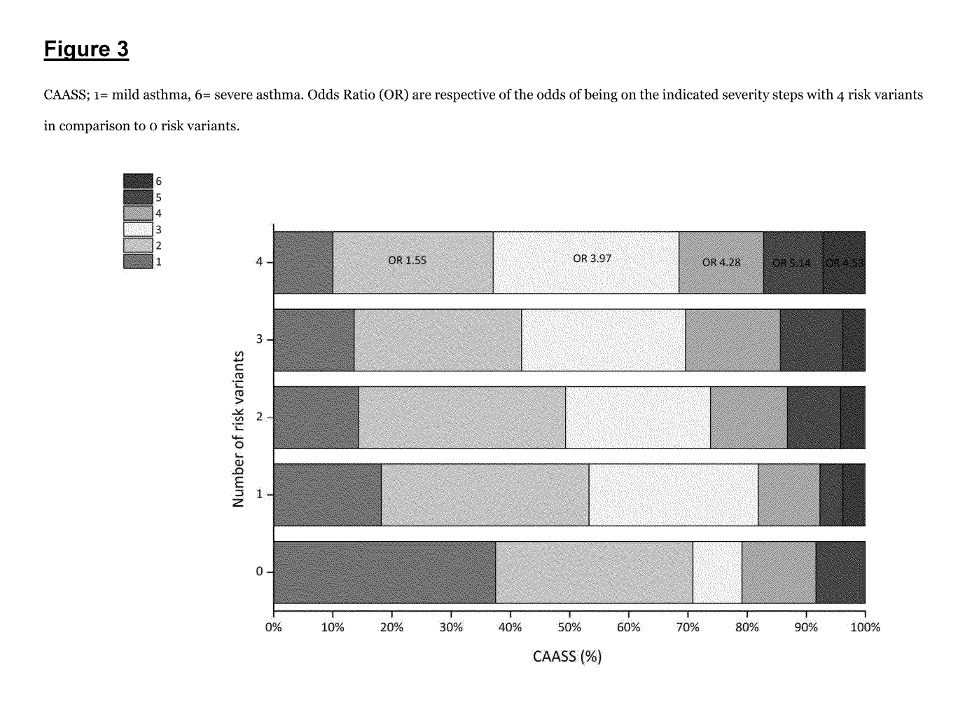

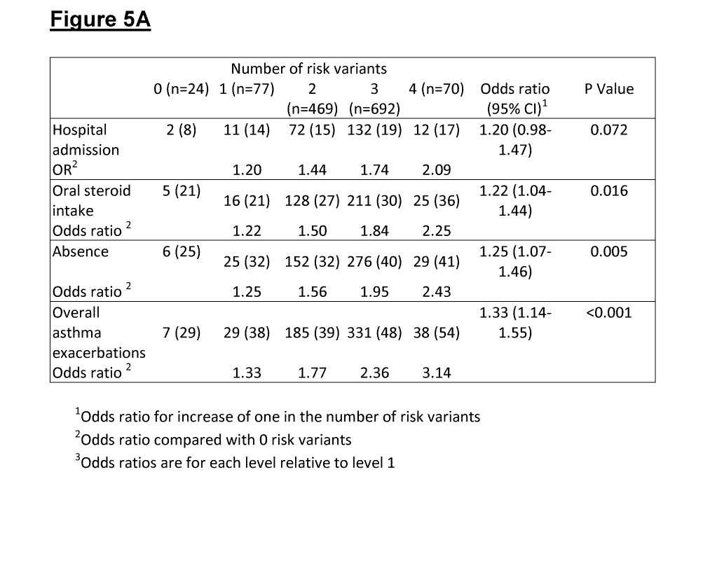

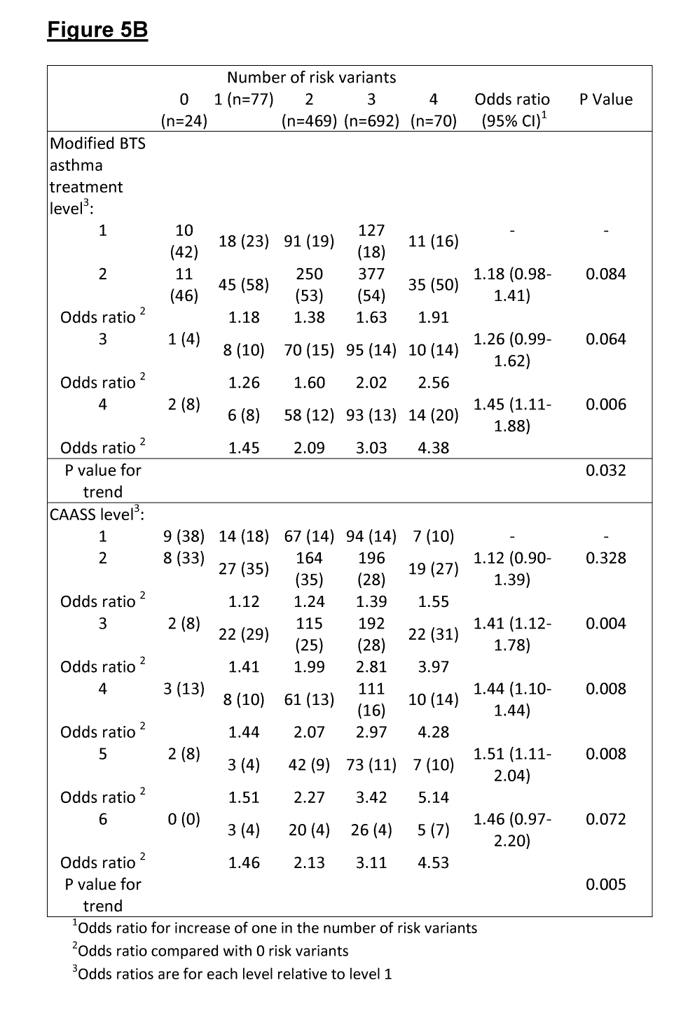

35.2% of the population (n=*469) possessed two, 52.0% (n=692) possessed three, while 5.3% of the population (n=70) possessed all four of the risk variants described above (FIG. 5A). The 4 SNPs showed a significant cumulative association with oral steroid requirements, school, college or work absences, overall asthma exacerbations, asthma treatment step and CAASS, after adjustment for age and sex, as shown in FIGS. 5A and 5B. Children and young adults who carried one, two, three or four of the risk variants had an increasing likelihood of severe asthma. This effect was observed for overall asthma exacerbations (odds ratio per genotypic step 1.33 (CI 1.14-1.55); P value for trend=2.05×10⁻⁴, as shown in FIG. 2) and Childhood and Adolescent Asthma Severity Score (CAASS) (odds ratio between CAASS level 1 and level 5=5.14; P value for trend=0.005, as shown in FIG. 3).

The inventors have therefore shown a surprising association between 4 SNPs (i.e. SEQ ID Nos. 1-4) and asthma exacerbations in a population of 1332 individuals, as shown in FIGS. 4A and 4B.

The finding of consistent associations between individual ‘at-risk’ variants that are functionally related led the inventors to explore the possibility of a cumulative risk of more severe asthma in young patients carrying more than one of these variants. They found that the SNPs had a highly significant cumulative association with asthma exacerbations (see FIG. 2 and FIGS. 5A and 5B), oral steroid use, absence from school/college/work, asthma treatment step (see FIGS. 5A and 5B) and CAASS (see FIG. 3 and FIGS. 5A and 5B). For example, there is an increase in the risk of having an asthma exacerbation with each stepwise increase in the number of risk variants. It was estimated that children and young adults with all four risk variants have an odds ratio of 5.14 for having the maximum CAASS score of 5, an odds ratio of 4.38 for being on the highest asthma treatment step 4, an odds ratio of 3.14 for experiencing an asthma exacerbation in the previous six months, an odds ratio of 2.43 for experiencing an exacerbation leading to absence from school, college or work in the previous six months, and an odds ratio of 2.25 for an exacerbation requiring oral steroids in the previous 6 months, in comparison to the population with none of the risk variants derived from the same population. The presence of an increasing number of ‘risk variants that may predispose to airway remodeling, and, in particular, the presence of all four ‘risk variants’ within this biological pathway, thus appears to exert a cumulative effect, increasing asthma severity.

Surprisingly, the differences between these patients are manifest over a relatively short time scale (only 6 months). The natural history of persistent, moderate to severe asthma spans several decades of life. It may thus be possible to use the combined information from the four SNPs (i.e. SEQ ID Nos. 1-4) to assess an individual patient's risk for developing severe asthma, and the likelihood of requiring more support from the healthcare services, over a prolonged course of time.

In summary, the inventors believe that the claimed invention is a first step towards developing a prognostic model for asthma severity in children and young adults. By using the combined model of four functionally related SNPs to predict an individual patient's risk of asthma severity, it may be possible to personalise treatment strategies, potentially reducing the burden of emergency healthcare, thus improving the standard of care and the quality of life in children and young adults with asthma. This strategy may also allow the testing of the efficacy of novel MMP 9 and MMP12 inhibitors such as AZD1236 in children and young adults with asthma who are most likely to benefit from such interventions, instead of trials in general populations with respiratory disease where such efficacy could be more difficult to demonstrate.

1. A method for providing a prognosis for an individual's asthma severity, the method comprising detecting, in a sample obtained from the individual, for the presence of a genetic polymorphism pattern in a matrix metalloproteinase 9 (MMP9) gene, wherein the polymorphism pattern is associated with asthma severity.

2. A method according to claim 1, wherein the polymorphism pattern in MMP9 comprises a MMP9 rs17576 SNP.

3. A method according to claim 1, wherein the method comprises detecting for the presence of the A-allele of the MMP9 rs17576 SNP.

4. A method according to claim 1, wherein the polymorphism pattern in MMP9 comprises a MMP9 rs6073983 SNP.

5. A method according to claim 1, wherein the method comprises detecting for the presence of the A-allele of the MMP9 rs6073983 SNP.

6. A method according to claim 1, wherein the method comprises detecting, in the sample, for the presence of a genetic polymorphism pattern in a matrix metalloproteinase 12 (MMP12) gene and/or a Chitinase 3-like 1 (CH13L1) gene, wherein the polymorphism pattern is associated with asthma severity.

7. A method according to claim 1, wherein the method comprises detecting for the presence of the G-allele of the MMP12 rs652438 SNP.

8. A method according to claim 1, wherein the method comprises detecting for the presence of the C-allele of the CHI3L1 rs4950928 SNP.

9. A method according to claim 1, wherein the method comprises detecting two, three or four genetic polymorphism patterns in MMP9, MMP12 or CH13L1.

10. A method for providing a prognosis for an individual's asthma severity, the method comprising detecting, in a sample obtained from the individual, for the presence of a genetic polymorphism pattern in at least two genes selected from the group consisting of: matrix metalloproteinase 9 (MMP9); matrix metalloproteinase 12 (MMP12) gene; and Chitinase 3-like 1 (CH13L1) gene, wherein the polymorphism pattern is associated with asthma severity.

11. A method according to claim 10, wherein the method comprises detecting, in the sample, for the presence of a genetic polymorphism pattern in MMP9; MMP12; and/or CH13L1, wherein the polymorphism pattern is associated with asthma severity.

12. A method according to claim 10, wherein the method comprises detecting for the G-allele the MMP12 rs652438 SNP, the C-allele of the CHI3L1 rs4950928 SNP, and either the A-allele of the MMP9 rs17576 SNP or the A-allele of the MMP9 rs6073983 SNP, or both.

13. A method according to claim 10, wherein the sample comprises a biological sample selected from blood, plasma, serum, spinal fluid, urine, sweat, saliva, tears, breast aspirate, prostate fluid, seminal fluid, vaginal fluid, stool, cervical scraping, cytes, amniotic fluid, intraocular fluid, mucous, moisture in breath, animal tissue, cell lysates, tumor tissue, hair, skin, buccal scrapings, nails, bone marrow, cartilage, prions, bone powder, ear wax, or combinations thereof.

14. A method according to claim 10, wherein the detecting step comprises use of at least one oligonucleotide operable to be used for amplification of genomic DNA encoding MMP9, MMP12 and/or CH13L1.

15. A method according to claim 14, wherein the oligonucleotide comprises a sequence selected from the group consisting of: SEQ ID No's 1-4, or a functional fragment or variant thereof.

16. An asthma prognostic kit for identifying an individual's genetic polymorphism pattern in a matrix metalloproteinase 9 (MMP9) gene, wherein the polymorphism pattern is associated with asthma severity, the kit comprising means for determining the presence of a genetic polymorphism pattern in an MMP9 gene.

17. An asthma prognostic kit according to claim 16 for identifying an individual's genetic polymorphism pattern in at least two genes selected from the group consisting of: a matrix metalloproteinase 9 (MMP9) gene; a matrix metalloproteinase 12 (MMP12) gene; and a Chitinase 3-like 1 (CH13L1) gene, wherein the polymorphism pattern is associated with asthma severity, the kit comprising means for determining the presence of at least two genetic polymorphism patterns in genes selected from the group consisting of: MMP9; MMP12; and CH13L1.

18. A kit according to claim 17, wherein the kit comprises means for detecting for: (i) the G-allele the MMP12 rs652438 SNP, (ii) the C-allele of the CHI3L1 rs4950928 SNP, (iii) or the A-allele of the MMP9 rs17576 SNP, or (iv) the A-allele of the MMP9 rs6073983 SNP.

19. The method of claim 1 further comprising:
administering, to the individual, a therapeutic agent that prevents, reduces or delays progression of asthma.

20. A method of identifying an allele which is associated with asthma severity, the method comprising identifying an allele which is in linkage disequilibrium with the MMP12 rs652438 SNP and/or CHI3L1 rs4950928 SNP and/or MMP9 rs17576 SNP and/or MMP9 rs6073983 SNP, wherein said SNP is associated with asthma severity.
